(19) United States
(12) Patent Application Publication
(10) Pub. No.: US 2023/0355164 A1
(43) Pub. Date: Nov. 9, 2023

(54) DIAGNOSING ORAL DYSFUNCTION AND METHODS OF TREATMENT

(71) Applicant: Nourished Young, LLC, Atlanta, GA (US)

(72) Inventor: Laura Avery Young, Atlanta, GA (US)

(21) Appl. No.: 18/129,839

(22) Filed: Apr. 1, 2023

Related U.S. Application Data
(60) Provisional application No. 63/326,934, filed on Apr. 4, 2022.

(51) Int. Cl. A63B 23/00; A61B 5/00

(52) U.S. Cl. CPC A61B 5/4205; A63B 23/00

(57) ABSTRACT

A tool for evaluating, diagnosing, and treating oral motor dysfunction in subjects such as infants.

### CROSS-REFERENCE TO RELATED APPLICATIONS

This application claims priority to U.S. Provisional Application Serial No. 63/326934, filed Apr. 4, 2022, the contents of which are incorporated by reference.

### FIELD OF THE INVENTION

The invention relates generally to evaluating oral motor function in a subject and related methods of treatment. More specifically, the invention relates to stimulating and scoring oral reflexes in a subject and prescribing a treatment protocol or treating any oral motor dysfunction.

### SUMMARY OF THE DISCLOSURE

Provided herein are methods of evaluating oral motor function, diagnosing an oral motor dysfunction, prescribing treatment options, and/or treating any diagnosed motor dysfunction. In some embodiments, the methods include an application of multiple treatments to address one or more issues with an oral reflex response.

In certain embodiments, the method involves treating an oral dysfunction in a subject, the method comprising: stimulating an oral reflex response in the subject; scoring the oral reflex response to produce a score; analyzing the score to diagnose an oral dysfunction; and providing a treatment protocol based on any diagnosed oral dysfunction. In some embodiments, the method further comprises treating the subject to reduce the symptoms of oral dysfunction.

In certain embodiments, the oral reflex is selected from gape, suck, thrust, right phasic bite, left phasic bite, left lateralization, and right lateralization. In some embodiments, the stimulating is performed by an evaluator.

In some embodiments, the method further comprises evaluating the subject’s oral posture. In some embodiments, the method further comprises evaluating the subject’s oral tolerance to touch.

In certain embodiments, the treatment protocol includes physical therapy to strengthen the neural connection between the subject’s brain and oral muscles. In some embodiments, the treatment protocol includes a frenotomy. In some embodiments, the treatment protocol includes physical therapy before and after the frenotomy.

In certain embodiments, the oral reflex score is less than 3.

In certain embodiments, the treatment protocol includes providing one to ten cues in a treatment session. In some embodiments, the treatment session is provided one to fifteen times per day.

### DETAILED DESCRIPTION

Methods of evaluating oral motor function in a human subject are provided herein. The methods include stimulating an oral reflex response and evaluating the response. In some embodiments, the methods include scoring the oral reflex response to produce a score. The score may be analyzed to produce a treatment protocol. The oral reflex response evaluation may be categorized under different tests identified as responsiveness, organization, range of motion, and toning (also referred to as “endurance”). In some embodiments, the evaluation includes tongue positioning of and/or aversion to touch of the tongue and/or palate. During the evaluation, a subject may be positioned on its back or at an angle suitable for feeding/nursing.

Before the present invention is described in greater detail, it is to be understood that this invention is not limited to particular cases described. It is also to be understood that the terminology used herein is for the purpose of describing particular embodiments only, and is not intended to be limiting, since the scope of the present invention will be limited only by the appended claims.

### Ranges and Definitions

Unless defined otherwise herein, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

As used herein, the singular forms “a”, “an”, and “the” include plural referents unless the context clearly dictates otherwise. Similarly, the phrase “one or more” means at least one and also includes plural referents, again unless the context clearly dictates otherwise.

As used herein, the terms “and/or” and “any combination thereof” and their grammatical equivalents may be used interchangeably. Solely for illustrative purposes, the following phrases “A, B, and/or C” or “A, B, C, or any combination thereof” can mean “A individually; B individually; C individually; A and B; B and C; A and C; and A, B, and C.”

As used herein, the term “about” in relation to a reference numerical value and its grammatical equivalents includes the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.” For example, the phrase “about 10, 15, 20,” and so on means “about 10, about 15, about 20,” and so on.

As will be apparent to those of skill in the art upon reading this disclosure, each of the individual embodiments described and illustrated herein has discrete components and features, which can be readily separated from or combined with the features of any of the other several cases without departing from the scope or spirit of the present invention. Any recited method can be carried out in the order of events recited or in any other order that is logically possible.

### Human Subjects

In some embodiments, the subject is a human. In some embodiments, the human is young, such as postpartum newborn, newborn, infant, baby, or toddler. The subject may also be categorized based on an age range. Age is a measurement of time starting at the moment of birth. For example, in some embodiments, the human subject ranges in age from about one hour old to about one year old. In some embodiments, the human subject may be a postpartum newborn ranging in age from about 0 minutes to about 48 hours, about 1 hour to about 48 hours, about 2 hours to about 48 hours, or about 4 hours to about 45 hours.

The postpartum newborn’s age may be about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes old. The postpartum newborn may be about one hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hour, about 9.5 hours, or about 10 hours old. In some embodiments, the human subject may be a postpartum newborn ranging in age from about 1 hour to about 5 hours, about 5 hours to about 10 hours, about 10 hours to about 15 hours, about 15 hours to about 20 hours, about 20 hours to about 25 hours, about 25 hours to about 30 hours, about 30 hours to about 35 hours, about 35 hours to about 40 hours, about 40 hours to about 45 hours, or about 40 hours to about 48 hours. The postpartum newborn may be about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5 hours, about 30 hours, about 30.5 hours, about 31 hours, about 31.5 hours, about 32 hours, about 32.5 hours, about 33 hours, about 33.5 hours, about 34 hours, about 34.5 hours, about 35 hours, about 35.5 hours, about 36 hours, about 36.5 hours, about 37 hours, about 37.5 hours, about 38 hours, about 38.5 hours, about 39 hours, about 39.5 hours, about 40 hours, about 40.5 hours, about 41 hours, about 41.5 hours, about 42 hours, about 42.5 hours, about 43 hours, about 43.5 hours, about 44 hours, about 44.5 hours about 45 hours, about 45.5 hours, about 46 hours, about 46.5 hours, about 47 hours, about 47.5 hours, or about 48 hours old.

In some embodiments, the newborn ranges in age of about 48 hours to about 12 weeks. The newborn may be about 2 days to about 12 weeks, about 3 days to about 12 weeks, about 4 days to about 12 weeks, about 5 days to about 12 weeks, about 6 days to about 12 weeks, about 1 weeks to about 12 weeks, about 1 week to about 8 weeks, about 1 week to about 4 weeks, about 4 weeks to about 8 weeks, about 4 weeks to about 12 weeks, about 8 weeks to about 12 weeks, or about 2 days to about 8 weeks. The newborn may be about 2 days, about 3 days, about 4 days, about 5 days, about 6 days, about 7 days, about 8 days, about 9 days, about 10 days, about 11 days, about 12 days, about 13 days, or about 14 days old. In some embodiments, the newborn may be about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks old. In some embodiments, the newborn may be about 1 week to about 3 months old.

In some embodiments, the human subject may be an infant. The infant may range in age from about 3 months to about 6 months, about 4 months to about 6 months, or about 5 months to about 6 months old. The infant may be about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, or about 28 weeks old.

In some embodiments, the human subject is a baby. The baby may range in age from about 6 months to about 12 months old, about 7 months to about 12 months, about 8 months to about 12 months, about 9 months to about 12 months, about 10 months to about 12 months, or about 11 months to about 12 months. The baby may be about 6 months, about 7 months, about 8 months, about 9 months, about 10 months, about 11 months, or about 12 months. In some embodiments, the baby may be about 6 months to about 7 months, about 6 months to about 8 months, about 7 months to about 9 months, about 6 months to about 9 months, about 8 months to about 10 months, about 9 months to about 12 months, or about 9 months to about 11 months old. In some embodiments, the baby may be about 6 months, about 6.5 months, about 7 months, about 7.5 months, about 8 months, about 8.5 months, about 9 months, about 9.5 months, about 10 months, about 10.5 months, about 11 months, about 11.5 months, or about 12 months old.

In some embodiments, the human subject is toddler. The age of a toddler may range from about 1 year to about 3 years, or about 2 years old. The toddler may be about 1 year about 1.5 years, about 2 years, about 2.5 years, or about 3 years old.

The human subject may show signs of an oral motor dysfunction, for example difficulty latching. In some embodiments, the human subject does or is suspected of having difficulty latching. The human subject may have or is suspected of having difficulty feeding. In some embodiments, the human subject is a potential candidate for a frenotomy operation.

### Oral Reflexes

The disclosure takes advantage of and applies recently discovered oral reflexes present in postpartum to toddler age humans. The oral reflexes remain present until the human subject is capable of choosing (i.e., volition) to move the muscles connected to the tongue, jaw, lips, and any muscle used to open the mouth and move the tongue.

The following seven oral reflexes have been discovered and may be used to evaluate and diagnose oral motor dysfunction in a subject. The oral reflexes are gape, thrust, suck, left lateralization, right lateralization, left phasic bite, and right phasic bite. The methods disclosed herein may evaluate and/or stimulate one or more of these oral reflexes.

Each oral reflex may occur instantaneously or may take several seconds to respond to a stimulus. For example, the reflex may take up to about two seconds, up to about three seconds, up to about four seconds, or up to about five seconds to respond. In some embodiments, the reflex occurs instantly, for example, between about 0.0001 seconds, about 1 second, about 1.5 seconds, about 2 seconds, about 2.5 seconds, about 3 seconds, about 3.5 seconds, about 4 seconds, about 4.5 seconds, or about 5 seconds after contact with a probe. If the response does not occur after about 5 seconds, about 6 seconds, about 7 seconds, or about 8 seconds, the neural connection between the brain and the muscle may not be present yet, and the subject may be prescribed a treatment protocol that includes exercises to develop and strengthen the neural connection between muscle and brain.

For any stimulating or evaluating of an oral reflex, a probe may be used. A probe may be a finger, or gloved finger, of an evaluator performing the stimulation or evaluation. The probe may be an object shaped to contact a subject’s face and mouth, such as, rounded edges. The probe may be a size that is small enough to fit into the subject’s mouth but not small enough to be swallowed, and may include a handle for the evaluator to grip. The probe may be composed of a molded plastic or metal. The probe may include sensors for collecting data similar to an evaluator’s finger. Where a probe includes sensors, it is designed to electrically communicate to a computer to record and analyze the data collected by the sensors.

An evaluator may include a medical professional such as a doctor, a dentist, a nurse, a lactation specialist, a midwife, etc. The evaluator may be a caregiver such as a guardian, doula, parent, etc. The evaluator may be trained or directed to perform the evaluation of the subject’s oral motor function. Additionally, the evaluator may also prescribe or carry out, in whole or in part, a prescribed treatment.

In some embodiments, to stimulate and/or evaluate gape, a probe may be applied to the chin of the subject. The probe may touch the chin. The probe may stroke down the chin starting under the lip and moving down to the edge of the chin. In some embodiments, the gape reflex is stimulated by contacting a probe to the bottom lip of a subject and moving downward toward the edge of the chin. When the gape response is properly stimulated, the subject opens its mouth into the shape like an “o.” Without being limited by theory, gape is useful for preparing the subject’s mouth to latch or receive a nipple of, for example, a breast or a bottle.

The thrust reflex, also referred to as the tongue thrust reflex, may be stimulated and/or evaluated by contacting the exterior aspect of the center of the bottom gumline and the inside of the bottom lip with a probe to stimulate the subject’s tongue to come forward towards the lips of the subject. The probe may touch the subject’s gums and lower lip. Without being limited by theory, thrust is useful for preparing the subject’s tongue to engage during latching, for example onto a nipple of a breast or a bottle.

The suck reflex may be stimulated and/or evaluated by contacting a probe to the hard palate of the subject’s mouth. In response, the subject’s tongue may curl up and around the probe in order to create pressure within the mouth, also known as sucking. It is understood that this pressure transports a liquid, gas, or semi-solid from an external environment into the mouth to be swallowed. Without being limited by theory, the suck reflex is thought to be the energy expended by the subject in order to bring nutrition into its mouth.

The left lateralization and right lateralization are reflexes that move the tongue to the left or the right in the mouth. These reflexes can be evaluated independently. In some embodiments, to stimulate and/or evaluate the left lateralization, a probe may contact the middle of the gum line and moves towards the left side along the gum line to the back of the subject’s mouth. The middle of the gum line is measured by about the front of the mouth. In some embodiments, the probe moves along the gum in a direction from the middle of the gum line to the left towards the back of the mouth stopping about a quarter to about half the distance between the middle gum line and the back of the mouth. The probe may be moved at a relatively slow pace for example about 0.5 cm per second. When the reflex responds, in some embodiments, the result is that the tongue sweeps over to the left side of the mouth. Without being limited by theory, this reflex is thought to help the subject position the nutrition source, for example a nipple of a breast or a bottle, into a more optimal position for the subject to feed.

In some embodiments, to stimulate and/or evaluate the right lateralization, a probe may contact the middle of the gum line and moves towards the right side along the gum line to the back of the subject’s mouth. In some embodiments, the probe moves along the gum in a direction from the middle of the gum line to the right towards the back of the mouth stopping about a quarter to about half the distance between the middle gum line and the back of the mouth. The probe may be moved at a relatively slow pace for example about 0.5 cm per second. When the reflex responds, in some embodiments, the result is that the tongue sweeps over to the right side of the mouth. Without being limited by theory, this reflex is thought to help the subject position the nutrition source, for example a nipple of a breast or a bottle, into a more optimal position for the subject to feed.

The left and right phasic bite are reflexes that move the mouth up and down. When each reflex responds properly, the mouth moves up and down in a rhythmic pattern. The left phasic bite and the right phasic bite may be evaluated independently of each other. To stimulate and or evaluate the left phasic bite reflex, a probe may contact the middle gum line and trace along the gum back to the left corner of the mouth. In some embodiments, when the probe reaches the left corner of the mouth, the jaw muscles will move the jaws down to form a bite and then up to stop the bite. To stimulate and or evaluate the right phasic bite reflex, a probe may contact the middle gum line and trace along the gum back to the right corner of the mouth. In some embodiments, when the probe reaches the corner of the mouth, the jaw muscles will move the jaws down to form a bite and then up to stop the bite. The reflex response may be one bite or several bites. In some embodiments, the evaluator is looking for a rhythmic pattern of biting after a set period of time.

### Tests

A Comprehensive Oral Reflex Evaluation (CORE) assessment is a full evaluation that assesses oral function in a subject, by evaluating Oral Tolerance to Touch, Oral Posture, and Oral Reflexes. In some embodiments, the assessments use the terms “Functional”, “Level 1”, “Level 2”, and “Level 3.” In some embodiments, the assessments use the terms “Expected”, “Slightly Reduced”, “Moderately Reduced”, and “Significantly Reduced.” The diagnostic tool and methods of treatment are not limited by the naming conventions used for the scoring categories. Rather, the assessments are evaluated on a set of criteria that can be divided into at least four categories as shown in Table 1.

TABLE 1

Scoring terms and points

| Scoring Term | | Points Assigned |
|---|---|---|
| Functional | Expected | 3 |
| Level 1 | Slightly Reduced | 2 |
| Level 2 | Moderately Reduced | 1 |
| Level 3 | Significantly Reduced | 0 |

### Oral Tolerance to Touch Assessment

The oral tolerance tests may be used to analyze whether or not the subject has an aversion to touch. The test includes the evaluation of the tolerance of touch of the subject’s palate and tongue and the duration of touch on the subject’s palate and tongue. The touch tolerance of the subject’s palate may be assessed by using a probe to touch the hard palate of the subject. The score is based on whether a probe can touch the entire hard palate with no aversion to initial touch, a probe can touch half of the hard palate without aversion to the initial touch, can touch the front one-fourth of the palate without aversion to the initial touch, or shows aversion with any touch inside the subject’s oral cavity. If the subject shows any aversion to touch on the hard palate, methods of treatment may be employed to reduce or eliminate the aversion to touch on the subject’s hard palate.

In some embodiments, the tolerance of touch on the subject’s tongue is assessed by using a probe to touch the subject’s tongue. The score is based on whether a probe can touch two-thirds of the subject’s tongue without aversion to the initial touch, can touch up to half of the tongue without aversion to the initial touch, can touch the front one-fourth of the tongue without aversion to the initial touch, or shows an aversion with any touch inside the oral cavity. If the subject shows any aversion to touch on the tongue, methods of treatment may be employed to reduce or eliminate the aversion to touch on the subject’s tongue.

In some embodiments, the duration of touch on the hard palate and/or tongue may be evaluated. The score is based on the duration of time that a probe can touch the hard palate and tongue, respectively. In some embodiments, the score is based on whether a probe can touch in a biologically safe area (i.e., “green zone”) for up to about 10 seconds with no aversion, shows aversion in the green zone after touching between about 5 seconds to about 10 seconds, shows aversion in the green zone after touching between 1 second to 5 seconds, or shows aversion in the green zone with a touch longer than 1 second. If the subject shows any aversion to touch in the green zone, methods of treatment may be employed to reduce or eliminate the aversion to the duration of touch on the tongue or hard palate, respectively.

Table 2 shows requirements for scoring of the Oral Tolerance Assessment and Table 3 shows the points based on the assessments discussed above and listed in Table 2. If all criteria can be evaluated, the Oral Tolerance Assessment will give a score out of 12 possible points as demonstrated in Table 3. Where criteria cannot be assessed because a subject is crying, for example, the evaluator may score that test as “N/A” or “--” and use a percentage and/or reduce the total number of overall points that are possible. For example, if the evaluator was not able to assess the duration of touch on the tongue, then the overall score would be out of 9 rather than 12.

TABLE 2

shows a scoring crib sheet for the Oral Tolerance Assessment.
ORAL TOLERANCE ASSESSMENT

| | | | | |
|---|---|---|---|---|
| | Expected | Slightly Reduced | Moderately Reduced | Significantly Reduced |
| Palate Location | Can touch entire hard palate with no aversion to initial touch | Can touch the ½ of the hard palate with no aversion to initial touch | Can touch the front ¼ of the palate with no aversion to initial touch | Shows aversion with any touch inside the oral cavity to initial touch |
| Tongue Location | Can touch ⅔ of the tongue with no aversion to initial touch | Can touch up to ½ of the tongue with no aversion to initial touch | Can touch the front ¼ of the tongue with no aversion to initial touch | Shows aversion with any touch inside of the oral cavity to initial touch |
| Duration | Can touch in the green zone for up 10+ seconds with no aversion | Shows aversion in the green one after touching for 5-10 seconds | Shows aversion in the green zone after touching for up to 5 seconds | Shows aversion in the green zone with a touch longer than 1 second |

TABLE 3

shows the points assigned based on the Oral Tolerance Assessment

| | Expected | Slightly Reduced | Moderately Reduced | Significantly Reduced | Not Assessed |
|---|---|---|---|---|---|
| Palate-Location | 3 | 2 | 1 | 0 | N/A |
| Palate-Duration | 3 | 2 | 1 | 0 | N/A |
| Tongue-Location | 3 | 2 | 1 | 0 | N/A |
| Tongue-Duration | 3 | 2 | 1 | 0 | N/A |

In some embodiments, where a subject shows any aversion to touch in the oral cavity, an evaluator may provide a diagnosis and methods of treatment. In some embodiments, an evaluator will prescribe a physical therapy to overcome or reduce a subject’s touch to oral aversion. In some embodiments, the subject must be treated for oral aversion before any other therapies can be employed to treat other oral dysfunctions. See the Methods Section for more details.

### Oral Posture Assessment

The oral posture test evaluates the posture, which is the location of the subject’s tongue at rest (i.e., before any stimuli), and range of motion, which measures how far the tongue remains suctioned to the hard palate as the subject’s jaw is gently opened. This test tells the evaluator where the tongue’s natural state of rest is located. The assessment can be performed in-part visually and in-part with a probe. For example, a subject may sleep with their mouth open. In that instance, an evaluator may be able to visualize how much contact is occurring between the subject’s tongue and hard palate. Additionally, a probe may be used to measure the contact between the subject’s tongue and hard palate. For posture, the top score of 3 is given when there is complete tongue to palate suction. The lowest score of 0 is given when there is no contact between the tongue and palate.

Range of motion for oral posture is evaluated when an evaluator gently opens the subject’s jaw from closed to fully open and notes at what point from closed to open the tongue loses suction to the hard palate, if at all. For range of motion, the highest score of 3 may be given when the subject’s tongue maintains suction to the palate through the full range of motion. The lowest score may be given when tongue loses suction less than one-fourth of the way through the full range of motion. Tables 4 and 5 provide more information on the Oral Posture Assessment. If all criteria can be evaluated, the Oral Posture Assessment will give a score out of 9 possible points as demonstrated in Table 5.

TABLE 4

shows the criteria for scoring posture and range of motion
ORAL POSTURE ASSESSMENT

| | | | | |
|---|---|---|---|---|
| | Expected | Slightly Reduced | Moderately Reduced | Significantly Reduced |
| Posture | Complete tongue to palate suction | Tip and some of the posterior tongue life to the palate | Tip of the tongue to palate | Tongue does not contact the palate |
| Range of Motion (ROM) | Maintains suction or cupping through the full ROM of the jaw | Maintains suction or cupping more than halfway through full ROM | Maintains suction or cupping less than halfway through full ROM | Maintains suction or cupping less than ¼ way through full ROM. |

TABLE 5

shows the scoring grid for the Oral Posture Assessment.

| | Expected | Slightly Reduced | Moderately Reduced | Significantly Reduced | Not Assessed |
|---|---|---|---|---|---|
| Tongue Posture at Rest | 3 | 2 | 1 | 0 | N/A |
| Tongue to Palate ROM | 3 | 2 | 1 | 0 | N/A |

When a subject shows a deficiency in Oral Posture, an evaluator may recommend a frenotomy after evaluating the subject’s oral reflexes.

### Oral Reflexes Assessment

The most comprehensive of the CORE assessment is the evaluation of Oral Reflexes. The overall high score that may be given in the Oral Reflexes Assessment is 78. The test may be used to evaluate the seven oral reflexes under the Responsiveness Test, Organization Test, Range of Motion Test, and Tonal Test (also referred to as the Endurance Test).

A Responsiveness Test evaluates the speed at which an oral reflex occurs after stimulation. One or more of the oral reflexes (i.e., gape, thrust, suck, left lateralization, right lateralization, left phasic bite, and right phasic bite) may be evaluated for speed of response. Without being limited by theory, the speed of the response may tell the evaluator how strong the connection is between the brain and muscle. During the evaluation the evaluator may score the speed of the response. In some embodiments, the Responsiveness Test is performed by an evaluator contacting the subject with a probe to elicit one or more oral reflex responses. The contact may be maintained for up to about 1 second, about 2 seconds, or about 3 seconds before repeating the contact. The evaluator may repeat the contact to stimulate the oral reflex response at least one more time, at least two more times, or at least three more times.

After contacting the subject with a probe to stimulate an oral reflex to evaluate the responsiveness, the evaluator scores the response. In some embodiments, the scoring is a measurement of the speed of responsiveness to the probe’s contact. For example, if the reflex response is observed within the first three seconds or so of contact, the evaluator marks the oral reflex as Functional (also referred to as “Expected”). When an oral reflex is found to be functional, the evaluator may allot 3 points for that reflex. Differing allotment of points for response times can be found below in Table 6. The probe’s contact may also be referred to as a “cue.”

TABLE 6

Exemplary Scoring grid for measuring Responsiveness in a subject
ORAL REFLEX ASSESSMENT - RESPONSIVENESS

| | Expected | Slightly Reduced | Moderately Reduced | Significantly Reduced | Not Assessed |
|---|---|---|---|---|---|
| Oral Reflex | Responds to initial reflexive cue within 3 seconds | Responds after one repeated cue | Responds after the second repeated cue | Does not respond within 3 repeated cues or shows other signs of aversion | N/A |
| Gape | 3 | 2 | 1 | 0 | -- |
| Thrust | 3 | 2 | 1 | 0 | -- |
| Suck | 3 | 2 | 1 | 0 | -- |
| R. Lateralization | 3 | 2 | 1 | 0 | -- |
| L. Lateralization | 3 | 2 | 1 | 0 | -- |
| R. Phasic Bite | 3 | 2 | 1 | 0 | -- |
| L. Phasic Bite | 3 | 2 | 1 | 0 | -- |

As illustrated above, a total score for the Responsiveness Test may range from 0 to 21 points. The score may be used to help the evaluator prescribe a treatment regimen, if needed.

An Organization Test evaluates the coordination between the brain and muscle, and is scored based on the number of reflexive responses out of five stimulation efforts. In some embodiments, the evaluator contacts the subject with a probe to stimulate an oral reflex. The probe maintains contact with the subject for up to about 1 second, about 2 seconds, or about 3 seconds and then the probe is removed from the subject. Then the cue is given again for up to about three more seconds before being removed. This test is performed until five separate cues have been given for a single oral reflex response. The evaluator then scores the test based on the number of oral reflex responses obtained after the five cues. For example, the evaluator may mark the oral reflex as Functional (also referred to as “Expected”) and assign a score of 3 if the oral reflex is produced five out of five times, once for each cue. Table 7 below illustrates how an evaluator may score the Organization Test.

TABLE 7

Exemplary Scoring grid for measuring Organization in a subject
ORAL REFLEX ASSESSMENT - ORGANIZATION

| | Expected | Slightly Reduced | Moderately Reduced | Significantly Reduced | Not Assessed |
|---|---|---|---|---|---|
| Oral Reflex | Reflex expressed 5/5 time after cue | Reflex expressed ⅘ times after cue | Reflex expressed 2-⅗ times after cue | Reflex expressed 0-⅕ times after cue | N/A |
| Gape | 3 | 2 | 1 | 0 | -- |
| Thrust | 3 | 2 | 1 | 0 | -- |
| Suck | 3 | 2 | 1 | 0 | -- |
| R. Lateralization | 3 | 2 | 1 | 0 | -- |
| L. Lateralization | 3 | 2 | 1 | 0 | -- |
| R. Phasic Bite | 3 | 2 | 1 | 0 | -- |
| L. Phasic Bite | 3 | 2 | 1 | 0 | -- |

As illustrated above, a total score for the Organization Test may range from 0 to 21 points. The score may be used to help the evaluator prescribe a treatment protocol, if needed.

A Range of Motion (ROM) Test evaluates the extent of the range of motion that the oral muscle can move. As described above, each oral reflex may be stimulated using a probe. The range of motion may be used to evaluate the muscles’ ability to move the tongue and/or jaw. In some embodiments, the evaluator stimulates an oral reflex response with a probe. When a muscle moves through the full range of motion in response to the stimulation, the evaluator may determine that the oral reflex response is Functional and may assign a score of 3. For example, when the oral reflex response can move at least halfway through the full range of motion, the evaluator may give a score of 2. In some embodiments, gape, thrust, right lateralization, and left lateralization can be evaluated for range of motion, because each of these assesses the ability of the subject’s tongue to transverse positions within the oral cavity or the ability of the mouth to form an “o” shape. Accordingly, suck, right phasic bite, and left phasic bite are not evaluated for ROM. An exemplary scoring grid for evaluating the range of motion is shown in Table 8.

TABLE 8

Exemplary Scoring grid for measuring the Range of Motion in a subject
ORAL REFLEX ASSESSMENT - ROM

| | Expected | Slightly Reduced | Moderately Reduced | Significantly Reduced | Not Assessed |
|---|---|---|---|---|---|
| Oral Reflex | Reflex pattern can move through the full ROM | Reflex can move at least ½ way through the full ROM | Reflex can move at least ¼ to about ½ way through the full ROM | Reflex can move less than ¼ way through the full ROM | N/A |
| Gape | 3 | 2 | 1 | 0 | -- |
| Thrust | 3 | 2 | 1 | 0 | -- |
| R. Lateralization | 3 | 2 | 1 | 0 | -- |
| L. Lateralization | 3 | 2 | 1 | 0 | -- |

As illustrated above, a score for the Range of Motion Test may range from 0 to 9 points. The score may be used to help the evaluator prescribe a treatment protocol, if needed.

A Tone Test (also referred to as “Endurance”) is used to evaluate the strength of the muscle to hold the reflex position or the strength of the muscle to repeat the reflexive motion. An evaluator may evaluate the tone for suck, right phasic bite, and left phasic bite. In some embodiments, the evaluator may contact the subject with a probe to stimulate the oral reflexive response for suck, right phasic bite, or left phasic bite. The probe may maintain contact to evaluate the ability of the oral reflex to repeat its movement rhythmically. The evaluator scores the tone of an oral reflex based on the number of rhythmic movements achieved before stopping. For example, an evaluator may determine that an oral reflex (i.e., suck, right phasic bite, or left phasic bite) is Functional if the reflexive movement occurs rhythmically 15 or more times during contact. If the oral reflexive response is Functional then the evaluator may assign it a score of 3. Table 9 provides an exemplary scoring grid for measuring the tone of suck and left or right phasic bite.

TABLE 9

Exemplary Scoring grid for measuring Tone in a subject
ORAL REFLEX ASSESSMENT - ENDURANCE REPEATING REFLEX

| | Expected | Slightly Reduced | Moderately Reduced | Significantly Reduced | Not Assessed |
|---|---|---|---|---|---|
| Oral Reflex | Reflex rhythmically repeats 15 or more times | Reflex rhythmically repeats between about 10 to 14 times | Reflex rhythmically repeats between about 5 to 9 times | Reflex rhythmically repeats between about 0 to 4 times | N/A |
| Suck | 3 | 2 | 1 | 0 | -- |
| R. Phasic Bite | 3 | 2 | 1 | 0 | -- |
| L. Phasic Bite | 3 | 2 | 1 | 0 | -- |

As illustrated above, a total score for Tone of these three reflexes may range from 0 to 9 points. The score may be used to help the evaluator prescribe a treatment protocol, if needed.

Separately, the evaluator may evaluate the subject’s strength for tongue thrust, right lateralization, left lateralization, and gape. When measuring the Endurance of these four reflexes, the test is slightly different because these reflexes are non-repeating reflexes. In some embodiments, the evaluator may contact the subject with a probe to stimulate the oral reflexive response for thrust, right lateralization, left lateralization, and gape. The probe may maintain contact to evaluate the ability of the subject to perform and hold the oral reflex. The evaluator scores the tone of an oral reflex based on how long the muscle can hold the reflexive position. For example, an evaluator may determine that an oral reflex (i.e., thrust, right lateralization, left lateralization, or gape) is Functional or Expected when the subject can hold the reflexive movement for more than 3 seconds. If the oral reflexive response is Functional then the evaluator may assign it a score of 3. Table 10 provides an exemplary scoring grid for measuring the tone of thrust, right lateralization, left lateralization, or gape.

TABLE 10

Exemplary Scoring Grid for measuring Tone in a subject
ORAL REFLEX ASSESSMENT - ENDURANCE NON-REPEATING REFLEX

| | Expected | Slightly Reduced | Moderately Reduced | Significantly Reduced | Not Assessed |
|---|---|---|---|---|---|
| Oral Reflex | Can hold tongue in the cued reflexive position for more than 3 seconds | Muscle held in the cued reflexive position for more than 2 seconds | Muscle held in the cued reflexive position for more than 1 second | Muscle held in the cued reflexive position for less than 1 second | N/A |
| Gape | 3 | 2 | 1 | 0 | -- |
| Thrust | 3 | 2 | 1 | 0 | -- |
| R. Lateralization | 3 | 2 | 1 | 0 | -- |
| L. Lateralization | 3 | 2 | 1 | 0 | -- |

As illustrated above, a score for Tone of these four oral reflexes may range from 0 to 12 points. The score may be used to help the evaluator prescribe a treatment protocol, if needed. When combining the scores for the Endurance Repeating Reflexes and Endurance Non-Repeating Reflexes a subject may receive a score out of 21 points.

Once all three assessments, Oral Tolerance, Oral Posture, and Oral Reflexes, are evaluated and scored, the total score may be from 0 to 96. However, if it is not possible to evaluate one or more of the reflexes, the total number of points may be reduced and a percentage calculated from the modified score to provide a normalized score. An evaluator may use the assessment to diagnose an oral dysfunction based on the scores, and provide a therapeutic treatment protocol such as sessions with a lactation consultant, physical therapy, or a frenotomy.

In an illustrative example, a study of 8 subjects were evaluated using the CORE Assessment before a frenotomy was performed (see results in Table 11) and then again post-frenotomy (see results in Table 12). The subjects were prescribed physical exercises to strengthen their oral motor function before and after the frenotomy. Tables 13 and 14 set forth the REFLEX ASSESSMENT scores within Tables 11 and 12.

TABLE 11

Subjects’ CORE assessment prior to a frenotomy

| BABY’S GENDER | DATE OF FRENOTOMY | AGE @ FRENOTOMY | AGE @ PRE/POST CORE EVALUATION | PRE-FRENOTOMY | | | | | |
|---|---|---|---|---|---|---|---|---|---|
| | | | | CORE ASSESSMENT SCORES | | | | | |
| | | | | TOLERANCE ASSESSMENT | POSTURE ASSESSMENT | REFLEX ASSESSMENT | TOTAL SCORE | % | CATEGORY |
| Female | Nov. 29, 2022 | 19 days old | 15 days old 29 days old | 12/12 | 2/6 | 46/72 | 60/90 | 66.67% | Moderately Reduced |
| Male | Aug. 02, 2022 | 1 mo 12 days | 22 days old 1 mos 28 days | 12/12 | N/A | 43/78 | 55/90 | 61.11% | Significantly Reduced |
| Female | May 03, 2022 | 1 mo 25 days | 1 mo 13 days 2 mos 24 days | 12/12 | N/A | 52/78 | 64/90 | 71.11% | Moderately Reduced |
| Male | Apr. 12, 2022 | 1 mo 15 days | 23 days 1 mon 24 days | 12/12 | N/A | 48/65 | 60/77 | 77.92% | Slightly Reduced |
| Male | Nov. 05, 2021 | 3 mos 5 days | 2 mos 14 days 3 mos 26 days | N/A | 3/6 | 42/78 | 45/84 | 53.57% | Significantly Reduced |
| Male | Feb. 01, 2022 | 2 mos 2 days | 1 mo 28 days 2 mos 22 days | 6/6 | 2/6 | 53/78 | 61/90 | 67.78% | Moderately Reduced |
| Female | Nov. 09, 2021 | 2 mos 6 days | 2 months 2 months 9 days | N/A | N/A | 29/54 | 29/54 | 53.70% | Significantly Reduced |
| Female | Mar. 01, 2022 | 1 mo 14 days | 1 month 12 days 3 months | 8/12 | 3/6 | 47/72 | 58/90 | 64.44% | Moderately Reduced |

TABLE 12

Subject’s CORE assessment after a frenotomy

| BABY’S GENDER | DATE OF FRENOTOMY | AGE @ FRENOTOMY | AGE @ PRE/POST CORE EVALUATION | POST-FRENOTOMY | | | | | |
|---|---|---|---|---|---|---|---|---|---|
| | | | | CORE ASSESSMENT SCORES | | | | | |
| | | | | TOLERANCE ASSESSMENT | POSTURE ASSESSMENT | REFLEX ASSESSMENT | TOTAL SCORE | % | CATEGORY |
| Female | Nov. 29, 2022 | 19 days old | 15 days old 29 days old | 12/12 | 3/6 | 59/72 | 74/90 | 81.94% | Slightly Reduced |
| Male | Aug. 02, 2022 | 1 mo 12 days | 22 days old 1 mos 28 days | 12/12 | N/A | 63/75 | 75/87 | 84.00% | Expected |
| Female | May 03, 2022 | 1 mo 25 days | 1 mo 13 days 2 mos 24 days | 8/12 | N/A | 60/78 | 68/90 | 76.92% | Moderately Reduced |
| Male | Apr. 12, 2022 | 1 mo 15 days | 23 days 1 mon 24 days | 12/12 | 2/6 | 59/78 | 71/90 | 75.64% | Slightly Reduced |
| Male | Nov. 05, 2021 | 3 mos 5 days | 2 mos 14 days 3 mos 26 days | N/A | N/A | 43/57 | 43/57 | 75.44% | Moderately Reduced |
| Male | Feb. 01, 2022 | 2 mos 2 days | 1 mo 28 days 2 mos 22 days | 6/6 | ⅔ | 60/75 | 68/84 | 80.00% | Slightly Reduced |
| Female | Nov. 09, 2021 | 2 mos 6 days | 2 months 2 months 9 days | N/A | N/A | 39/57 | 39/57 | 68.42% | Moderately Reduced |
| Female | Mar. 01, 2022 | 1 mo 14 days | 1 month 12 days 3 months | 6/12 | N/A | 51/72 | 57/84 | 70.83% | Moderately Reduced |

TABLE 13

The REFLEX ASSESSMENT of the 8 subjects pre- frenotomy.

| BABY’S GENDER | AGE @ FRENOTOMY | AGE @ PRE/POST CORE EVALUATION | RESPONSE CATEGORIES | PRE-FRENOTOMY | | | | | | | |
|---|---|---|---|---|---|---|---|---|---|---|---|
| | | | | ORAL REFLEX ASSESSMENT SCORES | | | | | | | |
| | | | | G | S | TE | RL | LL | RB | LB | CATEGORY % |
| Female | 19 days old | 15 days old 29 days old | RESPONSIVENESS | 3 | 3 | 0 | 3 | 3 | 3 | 3 | 85.7% |
| | | | ORGANIZATION | 3 | 3 | 1 | 3 | 3 | 3 | 3 | 90.5% |
| | | | ROM | 3 | 2 | 1 | 3 | 3 | N/A | N/A | 80.0% |
| | | | ENDURANCE | 3 | 1 | 0 | N/A | N/A | 0 | 0 | 26.7% |
| | | | Reflex Total | 12/12 | 9/12 | 2/12 | 9/9 | 9/9 | 6/9 | 6/9 | 53/72 |
| | | | Reflex % | 100% | 75% | 17% | 100% | 100% | 67% | 67% | 73.6% |
| Male | 1 mo 12 days | 22 days old 1 mos 28 days | RESPONSIVENESS | 3 | 3 | 1 | 2 | 2 | 3 | 3 | 90.5% |
| | | | ORGANIZATION | 3 | 3 | 1 | 2 | 2 | 3 | 3 | 90.5% |
| | | | ROM | 1 | 0 | 0 | 0 | 0 | N/A | N/A | 6.7% |
| | | | ENDURANCE | 2 | 3 | 0 | 0 | 0 | 1 | 1 | 33.3% |
| | | | Reflex Total | 9/12 | 9/12 | 2/12 | 4/12 | 4/12 | 7/9 | 7/9 | 42/78 |
| | | | Reflex % | 75% | 75% | 17% | 33% | 33% | 78% | 78% | 53.8% |
| Female | 1 mo 25 days | 1 mo 13 days 2 mos 24 days | RESPONSIVENESS | 3 | 3 | 3 | 3 | 3 | 3 | 3 | 100.0% |
| | | | ORGANIZATION | 3 | 3 | 3 | 3 | 3 | 3 | 3 | 100.0% |
| | | | ROM | 1 | 0 | 1 | 0 | 0 | N/A | N/A | 13.3% |
| | | | ENDURANCE | 3 | 3 | 1 | 1 | 0 | N/A | N/A | 53.3% |
| | | | Reflex Total | 10/12 | 9/12 | 8/12 | 7/12 | 6/12 | 6/6 | 6/6 | 51/72 |
| | | | Reflex % | 83% | 75% | 67% | 58% | 50% | 100% | 100% | 70.8% |
| Male | 1 mo 15 days | 23 days 1 mon 24 days | RESPONSIVENESS | 3 | 3 | N/A | 2 | 2 | 3 | 3 | 88.9% |
| | | | ORGANIZATION | 3 | 3 | N/A | 1 | 1 | 3 | 3 | 77.8% |
| | | | ROM | 2 | N/A | N/A | 0 | 0 | N/A | N/A | 22.2% |
| | | | ENDURANCE | 3 | 1 | N/A | 0 | 0 | 1 | 1 | 33.3% |
| | | | Reflex Total | 11/12 | 7/9 | N/A | 3/12 | 3/12 | 7/9 | 7/9 | 38/63 |
| | | | Reflex % | 92% | 78% | N/A | 25% | 25% | 78% | 78% | 60.3% |
| Male | 3 mos 5 days | 2 mos 14 days 3 mos 26 days | RESPONSIVENESS | 3 | 1 | 3 | 3 | 3 | 3 | 3 | 90.5% |
| | | | ORGANIZATION | N/A | 0 | N/A | N/A | N/A | 0 | 0 | 0.0% |
| | | | ROM | 0 | N/A | 2 | 1 | 2 | N/A | N/A | 41.7% |
| | | | ENDURANCE | N/A | N/A | N/A | N/A | N/A | N/A | N/A | N/A |
| | | | Reflex Total | 3/6 | ⅙ | ⅚ | 4/6 | ⅚ | 3/6 | 3/6 | 24/42 |
| | | | Reflex % | 50% | 17% | 83% | 67% | 83% | 50% | 50% | 57.1% |
| Male | 2 mos 2 days | 1 mo 28 days 2 mos 22 days | RESPONSIVENESS | 3 | 0 | 3 | 3 | 3 | 3 | 3 | 85.7% |
| | | | ORGANIZATION | 3 | 1 | 1 | 2 | 2 | 2 | 3 | 66.7% |
| | | | ROM | 3 | 0 | 2 | 2 | 2 | N/A | N/A | 60.0% |
| | | | ENDURANCE | 3 | 1 | 2 | 2 | 2 | 1 | 1 | 57.1% |
| | | | Reflex Total | 12/12 | 2/12 | 8/12 | 9/12 | 9/12 | 6/9 | 7/9 | 53/78 |
| | | | Reflex % | 100% | 17% | 67% | 75% | 75% | 67% | 78% | 67.9% |
| Female | 2 mos 6 days | 2 months 2 months 9 days | RESPONSIVENESS | 3 | 3 | 1 | 3 | 3 | 3 | 3 | 90.5% |
| | | | ORGANIZATION | N/A | 2 | N/A | N/A | N/A | 0 | 0 | 22.2% |
| | | | ROM | 3 | N/A | 1 | 1 | 1 | N/A | N/A | 50.0% |
| | | | ENDURANCE | N/A | 2 | N/A | N/A | N/A | N/A | N/A | 66.7% |
| | | | Reflex Total | 6/6 | 7/9 | 2/6 | 4/6 | 4/6 | 3/6 | 3/6 | 29/45 |
| | | | Reflex % | 100% | 78% | 33% | 67% | 67% | 50% | 50% | 64.4% |
| Female | 1 mo 14 days | 1 month 12 days 3 months | RESPONSIVENESS | 3 | 0 | 3 | 3 | 3 | 2 | 0 | 66.7% |
| | | | ORGANIZATION | 3 | 0 | 3 | 2 | 3 | 2 | 1 | 66.7% |
| | | | ROM | 3 | 0 | 2 | 2 | 1 | N/A | N/A | 53.3% |
| | | | ENDURANCE | 3 | 0 | 3 | 3 | 2 | 1 | 0 | 57.1% |
| | | | Reflex Total | 12/12 | 0/12 | 11/12 | 10/12 | 9/12 | 5/9 | ⅑ | 48/78 |
| | | | Reflex % | 100% | 0% | 92% | 83% | 75% | 56% | 11% | 61.5% |

TABLE 14

The REFLEX ASSESSMENT of the 8 subjects post- frenotomy.

| BABY’S GENDER | AGE @ FRENOTOMY | AGE @ PRE/POST CORE EVALUATION | RESPONSE CATEGORIES | POST-FRENOTOMY | | | | | | | |
|---|---|---|---|---|---|---|---|---|---|---|---|
| | | | | ORAL REFLEX ASSESSMENT SCORES | | | | | | | |
| | | | | G | S | TE | RL | LL | RB | LB | CATEGORY % |
| Female | 19 days old | 15 days old 29 days old | RESPONSIVENESS | 3 | 3 | 2 | 3 | 3 | 3 | 2 | 90.5% |
| | | | ORGANIZATION | 3 | 3 | 3 | 3 | 3 | 3 | 2 | 95.2% |
| | | | ROM | 3 | 2 | 1 | 3 | 3 | N/A | N/A | 80.0% |
| | | | ENDURANCE | 3 | 1 | 2 | N/A | N/A | 1 | 1 | 53.3% |
| | | | Reflex Total | 12/12 | 11/12 | 8/12 | 9/9 | 9/9 | 7/9 | 5/9 | 61/72 |
| | | | Reflex % | 100% | 92% | 67% | 100% | 100% | 78% | 56% | 84.7% |
| Male | 1 mo 12 days | 22 days old 1 mos 28 days | RESPONSIVENESS | 2 | 3 | 1 | 3 | 3 | 3 | 3 | 85.7% |
| | | | ORGANIZATION | 3 | 3 | 0 | 3 | 3 | 3 | 3 | 85.7% |
| | | | ROM | 3 | N/A | 0 | 3 | 3 | N/A | N/A | 75.0% |
| | | | ENDURANCE | 3 | 3 | 1 | 3 | 3 | 3 | 3 | 90.5% |
| | | | Reflex Total | 11/12 | 9/9 | 2/12 | 12/12 | 12/12 | 9/9 | 9/9 | 64/75 |
| | | | Reflex % | 92% | 100% | 17% | 100% | 100% | 100% | 100% | 85.3% |
| Female | 1 mo 25 days | 1 mo 13 days 2 mos 24 days | RESPONSIVENESS | 3 | 3 | 2 | 3 | 3 | 3 | N/A | 94.4% |
| | | | ORGANIZATION | 3 | 3 | 2 | 2 | 3 | 3 | 2 | 85.7% |
| | | | ROM | 2 | 1 | 2 | 2 | 2 | N/A | N/A | 60.0% |
| | | | ENDURANCE | 3 | 3 | 1 | 2 | 3 | 2 | 2 | 76.2% |
| | | | Reflex Total | 11/12 | 10/12 | 7/12 | 9/12 | 11/12 | 8/9 | 4/6 | 60/75 |
| | | | Reflex % | 92% | 83% | 58% | 75% | 92% | 89% | 67% | 80.0% |
| Male | 1 mo 15 days | 23 days 1 mon 24 days | RESPONSIVENESS | 3 | 3 | 2 | 1 | 3 | 3 | 3 | 85.7% |
| | | | ORGANIZATION | 3 | 3 | 2 | 1 | 1 | 3 | 3 | 76.2% |
| | | | ROM | 3 | 3 | 2 | 2 | 2 | N/A | N/A | 80.0% |
| | | | ENDURANCE | 3 | 3 | 1 | 3 | 3 | 1 | 1 | 71.4% |
| | | | Reflex Total | 12/12 | 12/12 | 7/12 | 7/12 | 9/12 | 7/9 | 7/9 | 61/78 |
| | | | Reflex % | 100% | 100% | 58% | 58% | 75% | 78% | 78% | 78.2% |
| Male | 3 mos 5 days | 2 mos 14 days 3 mos 26 days | RESPONSIVENESS | 3 | 1 | 2 | 3 | 3 | 3 | 3 | 85.7% |
| | | | ORGANIZATION | N/A | 1 | N/A | N/A | N/A | 3 | 2 | 66.7% |
| | | | ROM | 3 | 0 | 3 | 1 | 1 | N/A | N/A | 53.3% |
| | | | ENDURANCE | 3 | N/A | 2 | 3 | 3 | N/A | N/A | 73.3% |
| | | | Reflex Total | 9/9 | 2/9 | 7/9 | 7/9 | 7/9 | 6/6 | ⅚ | 43/57 |
| | | | Reflex % | 100% | 22% | 78% | 78% | 78% | 100% | 83% | 75.4% |
| Male | 2 mos 2 days | 1 mo 28 days 2 mos 22 days | RESPONSIVENESS | 3 | 3 | 3 | 2 | 2 | 3 | 3 | 90.5% |
| | | | ORGANIZATION | 3 | 3 | 3 | 2 | 1 | 3 | 3 | 85.7% |
| | | | ROM | 3 | N/A | 2 | 1 | 1 | N/A | N/A | 58.3% |
| | | | ENDURANCE | 3 | 2 | 1 | 2 | 2 | 3 | 3 | 76.2% |
| | | | Reflex Total | 12/12 | 8/9 | 9/12 | 7/12 | 6/12 | 9/9 | 9/9 | 60/75 |
| | | | Reflex % | 100% | 89% | 75% | 58% | 50% | 100% | 100% | 80.0% |
| Female | 2 mos 6 days | 2 months 2 months 9 days | RESPONSIVENESS | 3 | 2 | 2 | 2 | 3 | 3 | 3 | 85.7% |
| | | | ORGANIZATION | N/A | 3 | N/A | N/A | N/A | 0 | 0 | 33.3% |
| | | | ROM | 3 | 3 | 2 | 1 | 2 | N/A | N/A | 73.3% |
| | | | ENDURANCE | 3 | N/A | N/A | 1 | 2 | N/A | N/A | 66.7% |
| | | | Reflex Total | 9/9 | 8/9 | 4/6 | 4/9 | 7/9 | 3/6 | 3/6 | 38/54 |
| | | | Reflex % | 100% | 89% | 67% | 44% | 78% | 50% | 50% | 70.4% |
| Female | 1 mo 14 days | 1 month 12 days 3 months | RESPONSIVENESS | 3 | 0 | 1 | 3 | 3 | 3 | 3 | 76.2% |
| | | | ORGANIZATION | 3 | N/A | 1 | 1 | 3 | 3 | 3 | 77.8% |
| | | | ROM | 3 | 0 | 1 | 2 | 2 | N/A | N/A | 60.0% |
| | | | ENDURANCE | 3 | 0 | 1 | 2 | 3 | 2 | 2 | 61.9% |
| | | | Reflex Total | 12/12 | 0/9 | 4/12 | 8/12 | 11/12 | 8/9 | 8/9 | 51/75 |
| | | | Reflex % | 100% | 0% | 33% | 67% | 92% | 67% | 67% | 68.0% |

Additionally, the evaluator may have access to scoring grids and scoring criteria to use during the evaluation. The scoring grids also provide a place to keep track of each test’s score. One or more assessments may be used to prescribe a treatment protocol based on the diagnosed oral dysfunctions.

Methods of Treatment Following Evaluation

Once an evaluator has assessed one or more of Responsiveness Test, Organization Test, Range of Motion Test, and Tone Test for one or more of the seven oral reflex responses (i.e., gape, thrust, suck, left and right lateralization, and left and right phasic bite), the evaluator may analyze the scores and use them to diagnose the subject with an oral motor dysfunction. For example, a postpartum subject may be evaluated to determine if the subject may have issues latching and/or feeding. In some embodiments, a postpartum subject who shows signs of difficulty latching may be evaluated to determine which of the oral reflexes is less than functional. Based on the diagnosis, the evaluator may prescribe an appropriate treatment protocol.

For example, a subject who scores a 0, 1, or 2 on any row on any of the tests (i.e., responsiveness, organization, range of motion, or endurance), may be prescribed exercises to increase the neural connections between the brain and muscle. In some embodiments, any total score for a test ranging from 0 to 14 may be prescribed specific exercises to strengthen the connection between muscle and brain. Where an evaluator provides a score of 0, 1 or 2 for any row on any test for range of motion or tone, or any total score of 0 to 14 out of 21 possible points, the evaluator may prescribe specific exercises to increase the strength in the muscles. After the specific exercises are prescribed and applied, the subject’s score may gradually improve with a goal of scoring a 3 for each oral reflex response.

Depending on the oral reflex dysfunction (e.g., the low or non-responsiveness of the oral reflex to a cue), the evaluator may prescribe a set of exercises to improve the responsiveness or strength of the oral reflex response. In all instances of applying the exercise therapy, the subject should be calm and alert. The exercise may be provided up to about ten times per day (i.e., ten sessions). In some embodiments, the exercise is performed between two to ten, between three to nine, between four and eight, between five and ten, between five and seven, or between six to seven times. The exercise may performed about once about twice, about three, about four, about five, about six, about seven, about eight, about nine, about ten, or about eleven times per day. When a reflex is a single movement (i.e., gape, thrust, or left and right lateralization) the probe may be contacted to the subject and held for about three seconds or until the oral reflex response occurs, whichever occurs first. If the oral reflex does not occur within about three seconds, the probe is removed from contacting the subject, the evaluator pauses, and then the evaluator contacts the subject again in a way to attempt to stimulate the oral reflex response. For each exercise session, the cue should be provided at least five times. When a reflex is a rhythmic movement (i.e., suck or left and right phasic bite), the probe may contact the subject to stimulate the oral reflex response and begin counting the rhythmic reflexes (e.g., 1, 2, 3, 4, 5, etc. up to 15) until the subject reaches 16 or for up to sixteen seconds, whichever occurs first. For each exercise session, the cue may be repeated about four more times with pauses in between.

The specific exercises are similar to the steps to stimulate each oral reflex, with the goal being that the neurons are repeatedly stimulated to fire and move the muscle, and the muscles’ strength increases to hold a position longer. In some embodiments, a subject that scores a 0, 1, or 2 on one or more oral reflex responses on the Responsiveness Test or Organization Test may be prescribed an exercise therapy directed to strengthening the neural connection and/or muscles related to that one or more oral reflex response. For example, a subject with a score of 0, 1, or 2 on gape may be prescribed and treated with an exercise to first strengthen the connection between the brain and jaw muscles. In this instance, the subject may be given a cue up to about five or six times per session, during up to about ten sessions a day for about two weeks in order to increase the neural connection. In some embodiments, the subject progressively responds sooner and continuously to the cue over time. Additionally, the subject may be treated to increase the tone or range of motion of the gape reflex. In some embodiments, the subject may be contacted by a probe to stimulate gape and the contact may be maintained for about 2 to about 5 seconds in order to increase the strength of the muscle to hold the position of the gape (i.e., mouth open in an “o” shape). The maintained contact may be repeated up to five times in a single exercise session and the subject may be provided with the exercise up to fifteen times a day. Over the course of time, for example anywhere from a day to two weeks, the subject’s muscles may strengthen to increase the amount of time the subject can hold the gape open after the reflexive response is triggered. The focus on muscle strength and/or neural connection may also increase the range of motion performed by the muscle. Over time, the mouth may open wider in the gape position, or the tongue may thrust out further, or move from side to side a little further. A similar scenario can be applied to the other oral reflex responses where either the neural connection is strengthened, the muscles are strengthened to maintain the oral reflex response position, or both. In some embodiments, the subject’s tongue is never directly manipulated to move from side to side in the subject’s mouth by the evaluator. The ability of the tongue to move is one thing, but an evaluator physically moving the tongue from side to side does not increase the neural connection or the muscles necessary to move the tongue left or right.

In some embodiments, a subject may be a possible candidate for a frenotomy. It is understood that not all frenotomies are successful. An evaluator may use the disclosed evaluations to diagnose and determine if the subject is a good candidate for a frenotomy or if the frenotomy would cause more harm than good at that point. For example, when a subject’s score on a Responsiveness Test and/or Organization test is between 15 to 21, the subject may be recommended as a good candidate for a “tongue-tie” surgery, and a frenotomy may be performed to help with feeding issues. The high score tells the evaluator that the neural connection between the brain and the oral muscles is adequate and that the subject will have a high likelihood of success in learning how to latch and/or feed after the surgery. However, when a subject has a score between 8 and 14, the evaluator may recommend a therapy that comprises exercises and surgery, where the exercises are performed before surgery and/or after surgery. Finally, when a subject has a score between 0 and 7, the evaluator may recommend only exercises because the subject is not currently a good candidate for a frenotomy. Without being limited by theory, it is thought that when the neural connection between the brain and muscle is weak or non-existent, the subject will have a difficult time learning to feed even after the tongue tie surgery because the muscles are not capable of moving into the optimal positions to feed in response to a stimulus. Once the exercises are performed for a set time, for example, about two weeks, the subject may be reevaluated periodically within that two week period. At the end of the two weeks, if the subject’s score for responsiveness and/or organization has improved to a value ranging from 8 to 14, the subject may be recommended for surgery along with additional exercises to improve the remaining dysfunction in the lower scoring oral reflex responses. If the subject scores a 15 to 21, then the subject may be a good candidate for a frenotomy, and surgery may be performed.

In some embodiments, when a subject shows any aversion to touch in the oral cavity as assessed using the Oral Tolerance Assessment, an evaluator may diagnose the oral dysfunction and prescribe a therapy for treatment. In some embodiments, the treatment may include providing a cue to the subject’s hard palate, tongue, or both. The cue may be a single touch with the probe for about 1 second, about 2 seconds, about 3 seconds, about 4 seconds, about 5 seconds, about 6 seconds, about 7 seconds, about 8 seconds, about 9 seconds, or about 10 seconds to the hard palate or tongue. The treatment may include providing the cue at least one, at least twice, at least three, at least four, or at least five times in one treatment session. In some embodiments, the treatment session may be given at least once a day or at least once a week. In some embodiments, the treatment session may occur up to ten times a day or up to fifty times in one week.

In some embodiments, when a subject shows an aversion to touch in the oral cavity as assessed using the Oral Tolerance Assessment, an evaluator may diagnose the oral dysfunction and prescribe a therapy for treatment. In some embodiments, the treatment may include providing a cue to the subject’s hard palate, tongue, or both. The cue may be a single touch to the hard palate or tongue. In some embodiments, the cue may be a single touch with a probe that traverses over the front one-fourth of the palate or tongue, a single touch with a probe that traverses over half of the palate or tongue, or a touch with a probe over two-thirds of the tongue or over the entire hard palate of the subject. The treatment may include providing the cue at least one, at least twice, at least three, at least four, or at least five times in one treatment session. In some embodiments, the treatment session may be given at least once a day or at least once a week. In some embodiments, the treatment session may occur up to ten times a day or up to fifty times in one week.

Diagnostic Tool and Method of Treatment for Tongue-Tie in a Subject

In some embodiments, an alternative or additional assessment may be performed called a FAST assessment. In some embodiments, an evaluator, such as a dentist, may perform this evaluation before performing a frenotomy to determine if the subject is a good candidate for the surgery. The results of the FAST assessment along with a visual analysis of the subject’s tongue may tell the evaluator if a subject is a good candidate for the frenotomy that day.

One purpose of the FAST assessment is to determine the neural connection between a subject’s brain and oral muscles. If the connection is not adequate, then the frenotomy has an increased risk of being unsuccessful. Table 15 below provides the criteria and scoring grid for the FAST assessment.

TABLE 15

FAST Assessment scoring criteria and assigned points
FAST ASSESSMENT

| | Expected | Slightly Reduced | Moderately Reduced | Significantly Reduced |
|---|---|---|---|---|
| | Responds to the cue 3/3 times | Responds to the cue ⅔ times | Responds to the cue ⅓ times | Responds to the cue 0/3 times |
| Gape | 3 | 2 | 1 | 0 |
| Thrust | 3 | 2 | 1 | 0 |
| Suck | 3 | 2 | 1 | 0 |
| R. Lateralization | 3 | 2 | 1 | 0 |
| L. Lateralization | 3 | 2 | 1 | 0 |
| R. Phasic Bite | 3 | 2 | 1 | 0 |
| L. Phasic Bite | 3 | 2 | 1 | 0 |

In some embodiments, when an assessment results in a score between 18 to 21, the subject is a good candidate and is recommended for a frenotomy. When the subject scores a 17 or less, physical therapy including exercises to strengthen the reflex deficiencies may be prescribed to strengthen the neural connection before performing a frenotomy. The evaluator may prescribe physical therapy after the frenotomy, as well. In some embodiments, when the subject scores a 16 or less, the evaluator should prescribe physical therapy to strength the neural connection between the brain and the oral muscles. In some embodiments, an evaluator may prescribe a frenotomy when a subject scores a 17 or less if there is an emergent medical need or risk of premature feeding cessation. In these cases, physical therapy after the frenotomy may also be prescribed. Table 16 shows an evaluation of a FAST Assessment. The two subjects that scored a 15/21 and a 13/21 were referred for therapy to improve the neural connection between the brain and muscles prior to receiving the surgery. The other subjects all scoring 18 or above were referred for surgery that same day. The subject that scored a 15/21 was prescribed a treatment protocol to strengthen the connections for suck and thrust. While the subject that scored a 13/21 prescribed a treatment protocol to strengthen the connections for suck, thrust, right lateralization, and left lateralization.

TABLE 16

FAST Assessment of subject prior to surgery

| BABY’S GENDER | AGE @ FRENOTOMY | AGE @ PRE/POST CORE EVALUATION | AGE @ FAST SCREENING | FUNCTIONAL ASSESSMENT SCREENING TOOL FOR SURGICAL READINESS | | | | | | | |
|---|---|---|---|---|---|---|---|---|---|---|---|
| | | | | G | S | TE | RL | LL | RB | LB | FAST SCORE |
| Female | 19 days old | 15 days old 29 days old | 15 days old | 3 | 3 | 0 | 3 | 3 | 3 | 3 | 18/21 |
| Female | N/A | N/A | 1 mo 26 days | 3 | 0 | 0 | 3 | 3 | 3 | 3 | 15/21 |
| Female | N/A | N/A | 1 mo 2 days | 3 | 1 | 3 | 3 | 3 | 3 | 3 | 19/21 |
| Female | Pre-Frenotomy | FAST taken before frenotomy | 26 days old | 3 | 0 | 1 | 1 | 2 | 3 | 3 | 13/21 |
| Female | 2 mos 19 days | 3 mos 16 days @ Post Core Eval Only | 2 mos 17 days | 3 | 0 | 3 | 3 | 3 | 3 | 3 | 18/21 |
| Female | 5 mos 23 days | 4 mos 4 days @ Pre Core Eval Only | 3 mos 26 days | 3 | 0 | 3 | 3 | 3 | 3 | 3 | 18/21 |
| Female | 2 months 10 days | 1 mo 23 days @ Pre Core Eval Only | 8 days old | 3 | 1 | 3 | 3 | 2 | 3 | 3 | 18/21 |
| Female | 2 months 8 days | 2 months @ Pre Core Eval Only | 2 months old | 3 | 3 | 3 | 3 | 3 | 3 | 3 | 21/21 |

We claim:

1. A method of treating an oral dysfunction in a subject, the method comprising:
a. stimulating an oral reflex response in the subject;
b. scoring the oral reflex response to produce a score;
c. analyzing the score to diagnose an oral dysfunction; and
d. providing a treatment protocol based on any diagnosed oral dysfunction.

2. The method of claim 1, further comprising treating the subject to reduce the symptoms of oral dysfunction.

3. The method of claim 1, wherein the oral reflex is selected from gape, suck, thrust, right phasic bite, left phasic bite, left lateralization, and right lateralization.

4. The method of claim 1, wherein the stimulating is performed by an evaluator.

5. The method of claim 1, further comprising evaluating the subject’s oral posture.

6. The method of claim 1, further comprising evaluating the subject’s oral tolerance to touch.

7. The method of claim 1, wherein the treatment protocol includes physical therapy to strengthen the neural connection between the subject’s brain and oral muscles.

8. The method of claim 1, wherein the treatment protocol includes a frenotomy.

9. The method of claim 8, wherein the treatment protocol includes physical therapy before and after the frenotomy.

10. The method of claim 1, wherein the oral reflex score is less than 3.

11. The method of claim 1, wherein the treatment protocol includes providing one to ten cues in a treatment session.

12. The method of claim 11, wherein the treatment session is provided one to fifteen times per day.
